(19) United States
(12) Patent Application Publication
(10) Pub. No.: US 2025/0248684 A1
(43) Pub. Date: Aug. 7, 2025

(54) CONTRASTIVE REINFORCEMENT LEARNING-BASED NAVIGATION IN MEDICAL IMAGING

(71) Applicants: Siemens Medical Solutions USA, Inc., Malvern, PA (US); King's College London, London (GB)

(72) Inventors: Abdoul Aziz Amadou, London (GB); Vivek Singh, Princeton, NJ (US); Florin-Cristian Ghesu, Baiersdorf (DE); Young-Ho Kim, West Windsor, NJ (US); Alistair Young, London (GB); Puneet Sharma, Princeton Junction, NJ (US); Kawal Rhode, Croydon (GB)

(21) Appl. No.: 18/432,113

(22) Filed: Feb. 5, 2024

(51) Int. Cl. A61B 8/00; A61B 8/12; B25J 9/16; G06T 7/70

(52) U.S. Cl. CPC A61B 8/4263; A61B 8/12; A61B 8/4218; A61B 8/469; B25J 9/163; B25J 9/1697; G06T 7/70; G06T 2207/10132; G06T 2207/20081; G06T 2207/20084; G06T 2207/30004

(57) ABSTRACT

For movement of medical sensors for medical imaging, an artificial intelligence (AI) is trained using a contrastive reinforcement learning (CRL) framework. Simulation may be used to provide the training data. For training, the sampling for a given input instance in CRL may use trajectories simulated from different patients for better contrast. CRL, with or without the simulation feature and/or the sampling feature, may provide more generalized navigation, such as in interventional or diagnostic settings.

### BACKGROUND

The present embodiments relate to navigation in medical imaging, such as ultrasound imaging. Ultrasound is often used as an imaging modality for diagnostic and/or interventional purposes. Ultrasound allows clinicians to assess organs' functionality and structure in real-time. However, ultrasound scans vary in quality due to operator skill, and there is an increasing demand for scans, with not enough skilled sonographers to answer to that demand. Similarly, other types of medical imaging require operator skill and knowledge for proper positioning or navigation but may not have enough skilled technicians for operating the medical imager.

Artificial intelligence (AI) systems with navigation capabilities can help alleviate this problem. For instance, AI may be used to guide novice users during the scanning process, or, given a robotic interface, provide input to a robot for navigating to a given location. One requirement to build such an AI navigation system is to have enough navigation data, i.e., data that is acquired while clinicians are navigating to the anatomy of interest. Diagnostic data is typically acquired and stored during examination but does not include the navigation data. While collection of navigation data is possible, acquiring large datasets used for training AI is time-consuming and potentially requires the use of additional hardware to track the transducer position.

One solution is taught by Li, et al. “RL-TEE: Autonomous Probe Guidance for Transoesophageal Echocardiography Based on Attention-Augmented Deep Reinforcement Learning.” This approach uses a simulation environment, where a synthetic ultrasound image is generated from a CT scan given a transducer position. The AI then learns to navigate to desired views in the simulation environment. However, the AI as trained can only navigate to a single view at a time, hence each target view needs a dedicated network. This method lacks generalizability, making it less suitable for interventional cases, where clinicians might need to assess the location and status of different invasive devices or to look for structures that are not usually well defined in standard views.

### SUMMARY

By way of introduction, the preferred embodiments described below include methods, systems, and non-transitory computer readable storage media for movement of medical sensors for medical imaging. The AI is trained using a contrastive reinforcement learning (CRL) framework. Simulation may be used to provide the training data. For training, the sampling for a given input instance in CRL may use trajectories simulated from different patients for better contrast. CRL, with or without the simulation feature and/or the sampling feature, may provide more generalized navigation, such as in interventional or diagnostic settings.

In a first aspect, a method is provided for navigation of an ultrasound imaging transducer. A vector representing a goal for a position of the ultrasound imaging transducer is received. An action to reposition the ultrasound imaging transducer is generated based on the vector. The action is generated by a processor input of the vector to a contrastive reinforcement learned policy network. The contrastive reinforcement learned policy network outputs the action in response to the input of the vector. The action is then output on an output interface.

In a second aspect, a method is provided for training for movement of a medical sensor. A plurality of sample trajectories of the movement of the medical sensor relative to patients is obtained. A processor machine trains an agent to output the movement of the medical sensor. The agent machine is trained in a contrastive reinforcement learning framework with inputs from the sample trajectories. The agent as machine trained is stored.

In a third aspect, a system is provided for medical sensor navigation. A memory is configured to store a policy network machine trained in a contrastive reinforcement learning framework. An input is configured to receive a goal for medical imaging. A processor is configured to output an action to move the medical sensor towards the goal based on input of the goal and a current state of the medical sensor to the policy network, which policy network outputs the action in response to the input.

Other implementations or features of any of aspects 1-3 are summarized below as illustrative examples.

The present invention is defined by the following claims, and nothing in this section should be taken as a limitation on those claims. Features of one aspect or type of claim (e.g., method or system) may be used in other aspects or types of claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one implementation of a method for navigation of a medical sensor with a CRL agent;

FIG. 2 is a flow chart diagram of an implementation of the method for machine training for medical sensor movement with CRL;

FIG. 3 illustrates an example CRL framework for medical sensor movement; and

FIG. 4 is a block diagram of one implementation of a system for medical sensor navigation.

### DETAILED DESCRIPTION OF THE DRAWINGS AND PRESENTLY PREFERRED EMBODIMENTS

A navigation system for medical imaging is provided. CRL is used for autonomous navigation in medical imaging. An AI system is machine trained in navigating using the CRL framework. An agent is trained to navigate to a desired goal state. The AI is used for automated medical sensor movement (e.g., probe manipulation) or to provide real-time sensor manipulation guidance for technicians, such as during an interventional procedure. The agent may control a robot for automated movement (autonomous navigation) of the medical sensor or output instructions to allow a technician to perform the navigation. The AI outputs actions that will likely lead to imaging the input goal. These actions can be converted into a human-readable format and used to guide sonographers during a scan. Similarly, the output may be passed as input to a robotic system, which converts the outputs into physical motions to reach the goal.

Using the CRL framework, the resulting agent (policy network) can navigate to any desired anatomical landmark. Standard views are a subset of the views reachable by navigation by the agent. The agent may be used to track any anatomical landmark or device by use of an input goal defining that landmark or device. For example, the CRL framework allows a user to specify a goal in the form of an image. This allows a user to input a desired view to which the AI will navigate.

The AI based on the CRL framework may be used to navigate various medical sensors. For example, the moveable sensors of a computed tomography, single photon emission computed tomography, or x-ray imaging are moved using the AI. The example used herein is ultrasound. An ultrasound transducer is navigated using movements output by the AI. In a further example, the transducer is used for echocardiography, such as a trans-esophageal transducer (TEE) or a transducer on an intra-cardiac echocardiography (ICE) catheter. The AI generates outputs to move the TEE or ICE-based transducer to image anatomy and/or device of interest. While the examples below are focused on echocardiography, TEE imaging of the heart in particular, the CRL-based AI may be used in other ultrasound modalities, for other organs, or using other types of medical imaging sensors (e.g., x-ray source and/or detector, or gamma camera).

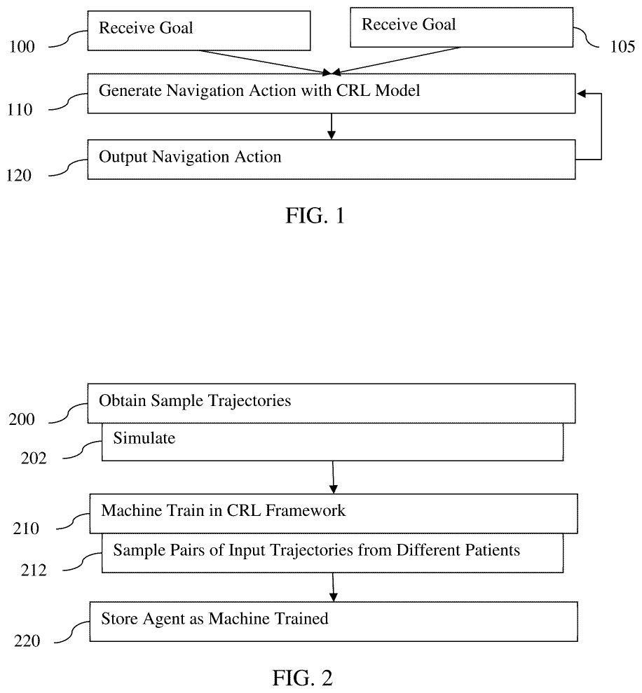

FIG. 1 is a flow chart diagram of one embodiment of a method for navigation of an ultrasound imaging transducer or other moveable medical imaging sensor. An agent, machine trained based on a CRL framework, uses an input goal and current state to move the sensor closer to the goal. The CRL framework used for training may use simulation and/or selective sampling for contrast in the CRL framework to train.

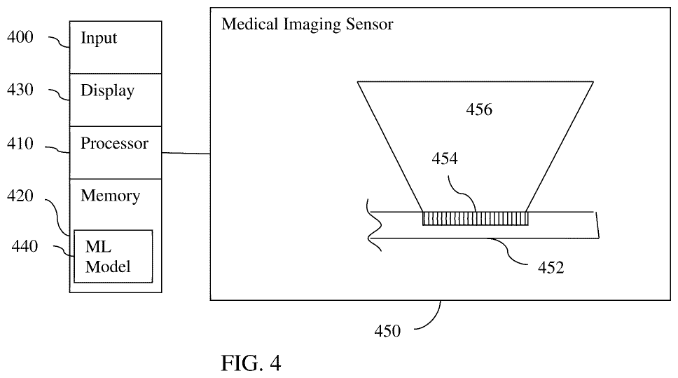

The method of FIG. 1 is implemented by the system of FIG. 4, a robotic navigation system, a medical imaging system, or another system. A processor receives and/or a memory stores a goal in act 100 and a current position in act 105, the processor generates one action or a sequence of navigation actions in act 110, and the processor outputs the generated navigation action or actions to a user or sonographer (e.g., to a display and/or speaker) and/or to a robotic navigation system.

Additional, different, or fewer acts may be provided. For example, acts for controlling the medical sensor or a robot to move the medical sensor are included. As another example, an act for defining or selecting the goal is provided. In yet another example, act 105 is not provided. The acts are performed in the order shown (top to bottom or numerical) or a different order.

In act 100, a processor receives a vector representing a goal for a position of the ultrasound imaging transducer. The processor receives the vector from memory, from user input, and/or from transfer over a computer network. Alternatively, the processor receives the vector by segmentation, landmark detection, or another image process of an image.

In act 105, the processor receives a vector representing a current position for the ultrasound imaging transducer. The processor receives the vector from memory, user input, and/or from transfer over a computer network. Alternatively, the processor receives the vector by segmentation, landmark detection, or another image process of an image.

One vector represents the current state. Another vector represents a goal state. The end or desired position of the transducer relative to patient anatomy or device is defined by the goal vector. The vector is one or more parameters that define the position. For example, the vector defines a field of view of the transducer relative to anatomy. In one approach, the vector is an image of anatomy and/or a device. The goal state is a vector representing an image from the transducer (i.e., what the image looks like and/or the anatomy in the field of view). The image may be a standard view or another (non-standard) view desired by a specific surgeon, practice, diagnosis, examination, and/or order. Example standard views may be apical two chamber, apical four chamber, or parasternal long axis, or another view. The image may represent a device instead of or in addition to the anatomy. For example, a view or image of an ablation catheter, needle, scalpel, or another device in the heart of the patient is the goal state.

In one approach, the physician or sonographer inputs a desired view from a library of views or another source. As another approach, the image is from a pre-operative or other medical scan, such as an ultrasound image of a desired view generated from a computed tomography scan. Other sources of the goal vector may be provided. The current state may be an image or measurements of the current position of the transducer.

In another implementation, the vector is one or more parameters defining the transducer position, such as the location and/or orientation of the transducer relative to a landmark. The field of view is defined by the transducer position. The goal state is a vector representing the desired transducer position. Other goal states or vectors representing the field of view, anatomy of interest, and/or imaging may be used.

In act 110, the processor generates an action to reposition the ultrasound imaging transducer. The goal and current states are used to determine a movement so that the transducer is repositioned closer to or further along a trajectory leading to the goal state or desired view as defined by the vector.

The action is a translation, rotation, or combination thereof in one, two, or three dimensions. The action is a change in the current state of the transducer and/or field of view, such as a change in position, location, and/or orientation. The action may be one in a sequence of steps to reach the desired goal. The action is generated based on the vector. The action avoids violating any boundary conditions, such as moving within a patient without passing through any walls (e.g., moving in the esophagus of the patient without positioning outside of the esophagus).

The processor generates the action by input of the vector to a policy network. The policy network is a neural network or other action model from reinforcement learning. The policy network was trained with CRL, so is a CRL policy network. In response to input of the goal vector and a current state vector, the CRL policy network (machine-trained agent) outputs the action. The current state vector is a position of the transducer or field of view, image, or other parameterization or representation of the field of view of the transducer in the current position. Other information may be input with the goal state and current state. In one implementation, the CRL policy network receives the goal vector or goal state as an image of a desired view and a current state as an image of the current field of view.

The CRL policy network was trained with an actor loss (reward) based on a critic network outputting the reward. The CRL framework includes both a critic network trained with a critic loss and an actor network trained with an actor loss. The actor loss is based on the output by the critic network. The critic network uses contrastive inputs as part of reinforcement learning of the actor policy to provide actions as steps. For example, the contrastive inputs are images sampled from different trajectories.

FIG. 2 is a flow chart diagram of one embodiment of a method for machine training for movement of a medical sensor. FIG. 2 is directed to training the CRL policy network in the CRL framework. The CRL policy network is trained as an agent for receiving a current state and goal state to output an action to move the current state closer to or further along a trajectory towards the goal state.

The method of FIG. 2 is implemented by a processor and memory. The memory stores sample trajectories, and the processor obtains samples of this training data for machine training by the processor. The trained agent is then stored in the memory or a different memory. In one approach, the system implementing the method of FIG. 2 is a computer, workstation, and/or server.

The method is implemented in the order shown (top to bottom or numerical) or another order. Additional, different, or fewer acts may be provided. For example, act 202 and/or act 212 are not provided.

In act 200, the processor obtains training data. A plurality of sample trajectories of the movement of the medical sensor (e.g., transducer) relative to one or more patients are obtained. For example, trajectories of moving an ultrasound transducer to one or more end states are acquired. Navigation data is acquired. The trajectories may be lists of possible transitions, possible current states, and possible end states (e.g., every voxel within an anatomical boundary is a possible current state and possible end state and ever adjacent voxel within the boundary is a possible transition. The trajectories may instead be paths to be followed with sampling intervals between a start point and an end point.

The training data is acquired by monitoring use. For example, navigation of transducers during a clinical study or from a library are accessed. In another approach, the training data is acquired by simulation in act 202 alone, or simulation in act 202 is used to add additional examples to samples from actual use.

For a simulation example, a computed tomography (CT) scan or magnetic resonance (MR) scan is performed on one or more patients (e.g., tens or hundreds of patients) for simulation in act 202. Each CT or MR scan is tomographically reconstructed into a representation of a volume of the scanned patient.

Segmentation and/or landmark detection may be used to define a region of possible positions of the medical sensor in each volume or representation. For example, the esophagus is segmented and defines a three-dimensional boundary for positioning a transducer within each patient.

For each possible position (location and/or orientation), a field of view is defined based on transducer parameters, such as transducer dimensions, number of elements, depth of scan, scan geometry, frequency of transmitted pulses, frequency of received echoes, and/or other information defining the field of view relative to the transducer. The CT data from the representation within the field of view is converted to ultrasound information. An ultrasound scan of the field of view is generated by simulation from the CT data. A synthetic ultrasound image is generated from the CT data given a position and/or transducer properties.

For each patient and given any boundary conditions (e.g., esophagus wall), a plurality of different trajectories from any one position to any other position may be generated. A plurality of trajectories may be provided for any pair of starting and ending positions. Similarly, a plurality of starting and ending pairs may be used to create respective sets of trajectories.

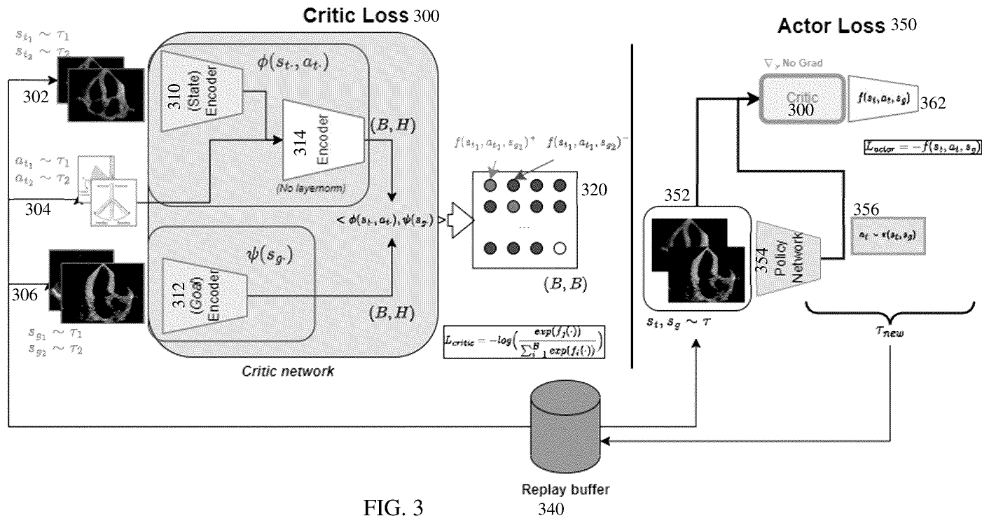

The different trajectories, whether from simulation or monitoring actual navigation, are used as training data in the CRL framework. FIG. 3 shows an example of the CRL framework. The trajectories, including or only being corresponding images labeled by position (e.g., location and orientation) and patient, are stored in a database or memory, such as the replay buffer 340.

In an example implementation, the trajectories t are stored in the replay buffer 340. Each transition from one imaging device position to the next position along each trajectory is saved as sₜ, aₜ, rₜ, sₜ₊₁ where sₜ is the current state aₜ timestep t, aₜ is the action applied aₜ step t, rₜ the reward received after taking aₜ, and sₜ₊₁ is the next state obtained after taking action aₜ in state sₜ. Other parameterizations may be used. The trajectories correspond to movement of the imaging transducer between views (i.e., different fields of view). The trajectory path is continuous or a series of two or more samples or points (e.g., voxels). The trajectory has a start and end, with no, one, two, or more waypoints or samples in between. The trajectory may be along a sequence of views. The imaging transducer will proceed from view-to-view along the trajectory. A sequence of views and corresponding movements of the imaging transducer from view to view is defined. The trajectory is a path of movement along the degrees of freedom of the transducer with any constraints (e.g., anatomical wall or boundaries). Various, all, or random possible trajectories are sampled to define the training data.

In act 210, the processor machine trains the agent or CRL policy network. The agent is trained to output movement (i.e., action) of the medical sensor. The agent outputs movement to position the transducer at or closer to the imaging goal. This training occurs in a CRL framework. Through this machine training, the agent learns to output an action given a current state and goal state. The training data used, type of training, and network architecture result in a unique or different agent and corresponding policy. Differences will result in different agents.

The training uses training data. The sample trajectories in the replay buffer 340 are used for training. Sample input state vectors (e.g., current image and goal image) and actions are selected and used for training. Since contrastive training is used, information from different trajectories are sampled. For each iteration in the optimization for training, two or more contrastive samples from different trajectories are used as inputs. For example, trajectories and corresponding state and actions are selected from the simulations from CT or MR imaging.

The sampling from the reply buffer 340 assists the contrastive learning. Pairs of inputs are sampled for a given instance of input to the critic 300 for contrastive learning. The critic 300 distinguishes between movement (action) along a desired trajectory to reach the goal and a different or not-desired trajectory. Pairs of current states, actions, and goals are input as sampled from different trajectories. To create a training batch, one transition is sampled from each trajectory in the replay buffer 340. For each state sₜ, a goal s_{g} is generated by sampling a future state from the discounted state occupancy measure using a geometric distribution Geom (1-γ). Other sampling may be used.

The trajectories being sampled for each pair may be from the same patient. In one implementation, the pairs of inputs are sampled in act 212 from different patients for a given iteration (input instance) in the optimization for machine learning. Each input instance for machine training uses states and actions from different patients. For a given instance, sampling trajectories from different patients more likely provides a greater distance or difference for contrast. For different instances of input for training, the transitions from the same two (just two) patients are used. Alternatively, trajectories from different pairs of patients (different combinations) may be used for different instances of sampling and input in the training.

For training the machine-learned model, the machine learning model arrangement is defined. The definition is by configuration or programming of the learning. The number of layers or units, type of learning, and other characteristics of the model are controlled by the programmer or user. In other embodiments, one or more aspects (e.g., number of nodes, number of layers or units, or type of learning) are defined and selected by the machine during the learning. Training data, including many samples of the input data, is used to train. In reinforcement learning, the output is an action or movement that is rewarded more for actions more likely to lead to the goal.

FIG. 3 shows an example model for the machine training of act 210 (FIG. 2). The model is a combination of neural networks formed as a critic 300 and actor 350. The critic 300 uses one loss, and the actor 350 uses another loss, so the critic 300 and actor 350 are sequentially or iteratively trained (e.g., fixing one when training the other). The critic 300 includes two or more encoders, such as the current state encoder 310, a state-action encoder 314, and a goal encoder 312. Additional, different, or fewer encoders (e.g., state encoder 310 incorporated into or being the state-action encoder 314) may be used. The actor 350 includes one or more encoders, such as the policy network 354, a neural network configured to encode a latent representation of the current state and goal state to output an action 356.

The encoders increase abstraction and decrease resolution. The final layer outputs a latent representation. Values for one or more features are output by the encoder in response to input data. The latent representation are values for features at an abstracted level relative to the input image data and/or vectors.

Any encoder may be used, such as neural network layers separated by down-sampling. Softmax, pooling, and/or other layers may be used. The encoders have one or more layers, such as ten or more layers, with any number of nodes in each layer. Each node may use any activation function. Any types of layers may be used. In one embodiment, a series of down sampling and convolutional layers are used, such as an encoder from a U-Net or image-to-image network. Max or other pooling layer layers may be included. Dropout layers may be used. DenseNet, convolutional neural networks, ResNet, fully connected neural network, and/or other network architectures may be used for an encoder. Any critic or actor networks for reinforcement learning may be used.

For training the actor 350, the output action 356 of the policy network 354 is input to the critic 300 to generate a probability as the actor loss 362. This actor loss is used as a reward in optimization of the learnable parameters of the policy network 356 to generate actions leading to the desired goal state from the current state. The policy network 354 implementing the agent is trained on the loss 362 generated from the critic 300.

For training the critic 300, CRL is used. Current states 302 (e.g., images) from two trajectories, actions 304 for the two trajectories, and goal states 306 (e.g., images) from the two trajectories are input. The goal encoder 312 receives the goal states 306 to output a goal vector or latent representation, which is a matrix of size B×H, where B is the number of (state sₜ, action aₜ, goal s_{g}) triplets sampled from the replay buffer, and H is the dimension of the latent representation. The state encoder 310 receives the current states 302 to output state information as a latent representation, which is combined with the actions 304 as input to the state-action encoder 314. The state-action encoder 314 outputs the state-action vector or latent representation of similar dimensions as the goal encoder 312. The critic 300 operates on the latent representation for the goal and the latent representation for the state-action pair, which is based on the movement or action from the actor 350. The critic network's 300 objective is to generate two latent representations, one of the goal using ψ(s_{g}) and another one of the state-action pair ϕ(sₜ, aₜ).

The state-action vector and goal vector are used to populate a matrix 320, representing probabilities for different state-actions to result in the desired goal. The state-action latent representation is combined with the actual goal representation (f (sₜ₁, aₜ₁, s_{g1})) as a positive example and with the goal from the other trajectory (f (sₜ₁, aₜ₁, s_{g2})) as a negative example. This matrix 320 is a dot product of the different trajectories used such that the dot product ϕ(sₜ, aₜₖ). (s_{gk}) between state, sₜₖ, action aₜₖ and goal s_{gk} coming from the same trajectory k is maximized, and the dot product between ϕ(sₜ, aₜₖ)·ψ(s_{gj}) or ϕ(sⱼ, aₜⱼ)·ψ(s_{gk}) with j≠k is minimized, where j is another trajectory, such as a trajectory from a different patient or the same patient but with a different goal from kThe matrix 320 is used to calculate the critic loss 362 for a given state, goal, and action triplet using a NCE loss. The NCE loss is applied on both rows and columns of the matrix 320, such that, on a row, the critic is penalized for large dot product values between ϕ(sₜ, aₜₖ) and goals ψ(s_{gj}), Where j≠k. Similarly, on a column, the critic is penalized for large dot product values between ϕ(sₜⱼ, aₜⱼ) and goals ψ(s_{gk}).

Other CRL frameworks may be used. Contrasting information is used for the critic, which is used to generate the reward or loss for training the actor given proposed actions by the actor. Other reinforcement learning arrangements providing this CRL may be used.

The image processor machine trains the critic 300 and the actor 350. The training learns values for learnable parameters (e.g., weights, connections, filter kernels, and/or other learnable parameters of the defined architecture). Reinforcement learning is used with contrastive training. A goal-conditioned policy is learned. The weights, connections, filter kernels, and/or other parameters are the features being learned. For example, convolution kernels are features being trained. Using the training data, the values of the learnable parameters of the model are adjusted and tested to determine the values leading to an optimum estimation of the output given an input. The critic 300 (encoders 310, 312, and 314) and actor 350 (policy network 354) are trained in an interleaved manner or sequentially using optimization. Using optimization, different values of the learnable parameters are tested across different samples of the training data to minimize a loss and/or maximize a reward. Any of various optimizations may be used for machine training the encoders and/or other networks. Adam, gradient descent, or another optimization is used to train.

In one approach, contrastive learning receives input pairs of positive and negative examples, such as from different trajectories. The contrastive learning learns representations so that positive pairs have similar representations and negative pairs have dissimilar representations. The contrastive representation is used in goal-conditioned reinforcement learning. The contrastive learning from the critic 300 estimates the Q-function (matrix 320) for training the policy network 354 using the Q-function as the reward 362. In this probabilistic perspective goal-conditioned reinforcement learning, the expected reward objective and associated Q-function are expressed as the probability (density) of reaching a goal in the future.

During training of the critic 300, the loss is minimized, or reward is maximized. The loss for the critic 300 is a NCE or infoNCE loss, such as a maximum dot product from a trajectory and minimum from different trajectories. In CRL, the critic 300 is trained to (1) maximize similarity between the state-action pair and the goal and (2) minimize similarity when sampled from a different trajectory. Through training over many instances of sampling, the critic loss aims aₜ: 1) maximizing the similarity between state-action pairs and goals from the same trajectories; and 2) minimizing the similarity when goals are sampled from different trajectories. The loss for calculating the maximization and/or minimization is NCE, InfoNCE, or another loss.

During training of the actor, the reward (e.g., Q-function) 362 from the critic 300 is maximized. The CRL policy network 354 is trained with the reward 362 being a probability of reaching desired result. The actor 350 is trained by giving the policy network 354 a state-goal pair (sₜ, s_{g}) where the goal is random or sampled in a pattern. The actor 350 (policy network 354) then outputs an action aₜ˜ π(sₜ, s_{g}), and the critic 300 takes the triplet (sₜ, s_{g}, aₜ) to produce a q-value 362 from the matrix 320. The reward (q-value) 362 indicates how likely it is that aₜ leads to s_{g} in the future. The critic is frozen during that pass, and the actor loss aims at maximizing that likelihood.

After training, the values of the learnable parameters of the architecture or model are set (fixed). These values may be updated in further training. Once established, the architecture (e.g., policy network 354 and/or encoders 310, 312, 314) and values of the parameters are ready for application. The critic 300 is not used in application, so the critic 300 may not be stored or packaged with the policy network 354 for distribution. The same policy network 354 may be copied and distributed for implementation by different computers, workstations, servers, and/or medical imaging systems.

In act 220 of FIG. 2, the policy network 354 is stored as a machine-trained agent. The machine-trained model or AI is stored. The learned weights, connections, kernels, and/or other values of the learnable parameters of the policy network 354 as learned are stored with the architecture or as part of the model. Where the policy network 354 is updated, refined, or retrained, the various resulting versions of the agent are stored.

The stored policy network 354 may be used or applied to generate the movement or navigation action in act 110 of FIG. 1. For application, the learned policy network 354 is used to generate an action 356 given input current state vector (e.g., image) and goal state vector (e.g., image). When deployed, the actor 300 receives as input an initial state so and a goal state s_{g} for a given patient. An action is generated and applied. After the action is applied, the current state sₜ is received while the goal state stays the same. Another action is generated. The actor 300 iteratively generates actions for different times aₜ˜ π(sₜ, s_{g}), once for each new observation sₜ₊₁. Once the action results in the current state matching the goal state (i.e., once the actions cause the current state to converge to the goal), the application is done. Any stopping criteria may be used to terminate, such as a threshold level of similarity between the current state and the goal state.

Due to the previous training, the learned actor 350 operates in a particular way to generate output actions given previously unseen inputs. The actor 350 (policy network 354) was trained in the CRL framework with the reward being a probability of reaching desired result, so attempts to achieve that goal in application. The actor was trained using a machine-trained critic. By sampling from different patients for each input instance in training the critic 300, the critic 300 forms a matrix for the loss, used to train the actor 350. The actor 350 acts in application based on the training of the critic 300. The actor 350 may act differently where the critic 300 was trained by sampling trajectories for the same patient in each input instance in training the critic 300. Where the training data is from simulation, a greater diversity of trajectories may result. The actor 350 may operate acceptably in a wider range of circumstances by having been trained by the critic 300, which is trained over the wider range from the simulation. The critic 300 was trained with a loss to maximize similarity between state-action pairs and goals and minimize similarity between goals from different trajectories. The actor 350 was trained based on that critic 300 so operates differently due to the contrastive loss in the CRL framework.

In act 120, the action or actions are output to an output interface. The processor generates the output action. The movement or navigation is output.

The action may be converted or formatted for a particular output device. The output is on an output interface, such as a user interface (e.g., a display, haptic feedback, and/or speaker). The sonographer or technician is instructed to move the transducer in translation and/or rotate. A sequence of such movements from the policy network 354 and implemented by the user places the transducer at the goal.

In another implementation, the output is to a robotic navigation system. The output interface is a buffer, bus, computer network, or memory. The actions are output to a different processor or to the same processor executing a robotic control process. The actions are converted (e.g., using kinematics) to controls for moving the transducer by the robotic system.

The feedback arrow from act 120 to act 110 represents feedback of a new state after movement of the transducer and/or patient (i.e., movement of the transducer relative to the patient). The robotic system and/or user moves the transducer (or another medical sensor) based on an output action. This results in a new current state vector, which is provided back for input as the current state with the goal state to the policy network 354, which generates a new action. In other approaches, the actor generates a sequence of actions in response to one input, which sequence may or may not be updated based on change in the current state.

During navigation under the influence of the output actions, images may be generated. These navigation images are displayed. When the goal is reached, the images may be used for diagnosis and/or to monitor an interventional procedure. The goal may be changed for different parts of the procedure, so the goal state is changed. Since the CRL framework was used, the trained policy network 354 more likely handles change in the goal and current state so that actions may continue to be generated to lead the current state to the current goal.

FIG. 4 is a block diagram of one embodiment of a system for medical sensor navigation. A CRL model, such as a learned agent or policy network as a machine-learned model 440, is executed to generate actions for navigating the medical sensor. In this example, the medical sensor is an ultrasound transducer array 454 on a TEE probe 452. Other medical sensors, such as an x-ray source and/or detector on a C-arm, may be used. Any moveable imaging sensor may be controlled to move based on output by the agent or trained model 440.

The system includes a processor 410, memory 420, input 400, and display 430. The processor 410, memory 420, input 400, and/or display 430 are part of a computer, workstation, or server and/or are part of a medical imaging system or robotic navigation system. Additional, different, or fewer components may be provided. For example, the input 400 and/or display 430 are not provided.

The input 400 is a device, such as an input of a graphical user interface. A keyboard, mouse, touchpad, touchscreen, trackball, buttons, sliders, knobs, and/or other input devices may be used. The input allows user interaction with the processor 410, such as for inputting a goal (e.g., selecting or inputting a desired view). The input 400 is configured to receive the goal for medical imaging. In an alternative implementation, the input 400 is a computer interface for receiving the goal over a computer network or computer communications.

The memory 420 is a non-transitory memory, such as a removable storage medium, a random-access memory, a read only memory, a memory of a field programmable gate array, a cache, and/or another memory. The memory 420 is configured by a processor, such as the processor 410, to store the machine-learned model 440 as previously machine trained in the CRL framework. Other information, such as current state vectors, actions, and/or goal state vectors may be stored. Any information used and/or generated by the processor 410 in navigation may be stored in the memory 420. Alternatively, or additionally, the memory 420 stores instructions executable by the processor 410.

The processor 410 is a general processor, application specific integrated circuit, integrated circuit, digital signal processor, field programmable gate array, artificial intelligence processor, tensor processor, graphics processing unit, and/or another processor for implementing or applying the machine-learned model 440 (e.g., policy network 354). The processor 410 is configured by design, hardware, firmware, and/or software to apply the machine-learned model 440. Multiple processors may be used for sequential and/or parallel processing as the processor 410. The instructions from the memory 420, when executed by the processor 410, cause the processor 410 to generate actions to navigate to the input goal.

In one implementation, the processor 410 is configured to output an action to move the medical sensor (e.g., transducer 454) towards the goal based on input of the goal and a current state of the medical sensor to the policy network (machine-learned model 440). The positions, images, and/or fields of view 456 are used as state input vectors. In response to input, the processor 410 uses the machine-learned model 440 to outputs an action. Since the machine-learned model was trained in a CRL framework or using contrastive learning with reinforcement learning, the machine-learned model 440 may provide actions appropriate in various situations. Since the machine-learned model 440 was trained using trajectories with inputs sampled from different patients for a same iteration in optimization of a critic network of the CRL framework, the machine-learned model 440 may provide actions appropriate in various situations. An even greater range of generalization may be provided by having trained using trajectories created from simulation.

The display 430 is a display screen, such as a liquid crystal display or monitor. The display 430 may include icons, graphics, or other imaging, such instructions for how (movement vector such as translation direction, rotation direction, and/or magnitude) to move the transducer 454 and/or probe 452. The instructions on the display 430 guide or navigate so that the field of view 456 and corresponding image are of the goal.

The display 430 is configured to display images from the ultrasound transducer 454 or another moveable medical sensor. The images are of views while navigating to the desired view and/or are of the desired view.

Various illustrative examples are provided below. These examples may be provided in various combination. Features or aspects used for one type (e.g., method or system) and/or context (e.g., use as trained or training) may be used in the other type and/or context.

Illustrative example 1: a method for navigation of an ultrasound imaging transducer, the method comprising: receiving a vector representing a goal for a position of the ultrasound imaging transducer; generating an action to reposition the ultrasound imaging transducer based on the vector, the action generated by a processor input of the vector to a contrastive reinforcement learned policy network, the contrastive reinforcement learned policy network outputting the action in response to the input of the vector; and outputting the action.

Illustrative example 2: the method of illustrative example 1, wherein receiving comprises receiving as a user input.

Illustrative example 3: the method of any of illustrative examples 1 or 2, wherein receiving comprises receiving the vector as an image of anatomy and further comprising receiving another vector representing a current position of the ultrasound imaging transducer, wherein generating comprises generating the action by input of the vector representing the goal and the vector representing the current position to the reinforcement learned policy network.

Illustrative example 4: the method of illustrative example 3, wherein receiving the image comprises receiving the image of the anatomy as a standard view.

Illustrative example 5: the method of any of illustrative examples 1-4, wherein receiving comprises receiving the vector as a location and orientation of the ultrasound imaging transducer.

Illustrative example 6: the method of any of illustrative examples 1-5, wherein generating comprises generating with the contrastive reinforcement learned policy network comprising a neural network trained with an actor loss based on a critic network outputting a reward.

Illustrative example 7: the method of illustrative example 6, wherein generating comprises generating with the contrastive reinforcement learning policy network having been trained with the reward being a probability of reaching desired result.

Illustrative example 8: the method of any of illustrative examples 6 or 7, wherein generating comprises generating with the contrastive reinforcement learning policy network having been trained with the critic network comprising first and second encoders configured to receive states sampled from just two different patients to form a matrix for critic loss.

Illustrative example 9: the method of any of illustrative examples 6-8, wherein generating comprises generating with the contrastive reinforcement learning policy network having been trained with the critic network comprising first and second encoders configured to receive states from different trajectories for a same patient.

Illustrative example 10: the method of any of illustrative examples 6-9, wherein generating comprises generating with the contrastive reinforcement learning policy network and critic network having been trained using trajectories from simulation from computed tomography or magnetic resonance imaging.

Illustrative example 11: the method of any of illustrative examples 6-10, wherein generating comprises generating with the contrastive reinforcement learning policy network having been trained with the critic network having a critic loss to maximize similarity between state-action pairs and goals and minimize similarity between goals from different trajectories.

Illustrative example 12: the method of any of illustrative examples 1-11, wherein outputting comprises outputting the action comprising instructions to move the ultrasound imaging transducer.

Illustrative example 13: a method for training for movement of a medical sensor, the method comprising: obtaining a plurality of sample trajectories of the movement of the medical sensor relative to patients; machine training, by a processor, an agent to output the movement of the medical sensor, the agent machine trained in a contrastive reinforcement learning framework with inputs from the sample trajectories; and storing the agent as machine trained.

Illustrative example 14: the method of illustrative example 13, wherein the medical sensor comprises an ultrasound transducer, and wherein machine training comprises training the agent to output the movement to position the ultrasound transducer aₜ an imaging goal.

Illustrative example 15: the method of any of illustrative examples 13 or 14, wherein obtaining comprises obtaining from simulation.

Illustrative example 16: the method of illustrative example 15, wherein machine training comprises machine training the agent using pairs of input trajectories sampled from different patients for a given iteration in an optimization.

Illustrative example 17: the method of any of illustrative example 13-16, wherein machine training comprises machine training with the contrastive reinforcement learning framework comprising a policy network implementing the agent trained on a critic loss where the critic loss is from a critic network operating on a first latent representation for a goal and a second latent representation for a state-action pair, the state-action pair based on the movement.

Illustrative example 18: the method of illustrative example 17, wherein machine training comprises machine training with the critic network is trained to maximize similarity between the state-action pair and the goal and minimize similarity when sampled from a different trajectory.

Illustrative example 19: a system for medical sensor navigation, the system comprising: a memory configured to store a policy network machine trained in a contrastive reinforcement learning framework; an input configured to receive a goal for medical imaging; and a processor configured to output an action to move the medical sensor towards the goal based on input of the goal and a current state of the medical sensor to the policy network, which policy network outputs the action in response to the input.

Illustrative example 20: the system of illustrative example 19 wherein the policy network was trained using trajectories with inputs sampled from different patients for a same iteration in optimization of a critic network of the contrastive reinforcement learning framework, the trajectories created from simulation.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for navigation of an ultrasound imaging transducer, the method comprising:
receiving a vector representing a goal for a position of the ultrasound imaging transducer;
generating an action to reposition the ultrasound imaging transducer based on the vector, the action generated by a processor input of the vector to a contrastive reinforcement learned policy network, the contrastive reinforcement learned policy network outputting the action in response to the input of the vector; and
outputting, by an output interface, the action.

2. The method of claim 1, wherein receiving comprises receiving as a user input.

3. The method of claim 1, wherein receiving comprises receiving the vector as an image of anatomy and further comprising receiving another vector representing a current position of the ultrasound imaging transducer, wherein generating comprises generating the action by input of the vector representing the goal and the vector representing the current position to the reinforcement learned policy network.

4. The method of claim 3, wherein receiving the image comprises receiving the image of the anatomy as a standard view.

5. The method of claim 1, wherein receiving comprises receiving the vector as a location and orientation of the ultrasound imaging transducer.

6. The method of claim 1, wherein generating comprises generating with the contrastive reinforcement learned policy network comprising a neural network trained with an actor loss based on a critic network outputting a reward.

7. The method of claim 6, wherein generating comprises generating with the contrastive reinforcement learning policy network having been trained with the reward being a probability of reaching desired result.

8. The method of claim 6, wherein generating comprises generating with the contrastive reinforcement learning policy network having been trained with the critic network comprising first and second encoders configured to receive states sampled from just two different patients to form a matrix for critic loss.

9. The method of claim 6, wherein generating comprises generating with the contrastive reinforcement learning policy network having been trained with the critic network comprising first and second encoders configured to receive states from different trajectories for a same patient.

10. The method of claim 6, wherein generating comprises generating with the contrastive reinforcement learning policy network and critic network having been trained using trajectories from simulation from computed tomography or magnetic resonance imaging.

11. The method of claim 6, wherein generating comprises generating with the contrastive reinforcement learning policy network having been trained with the critic network having a critic loss to maximize similarity between state-action pairs and goals and minimize similarity between goals from different trajectories.

12. The method of claim 1, wherein outputting comprises outputting the action comprising instructions to move the ultrasound imaging transducer.

13. A method for training for movement of a medical sensor, the method comprising:
obtaining a plurality of sample trajectories of the movement of the medical sensor relative to patients;
machine training, by a processor, an agent to output the movement of the medical sensor, the agent machine trained in a contrastive reinforcement learning framework with inputs from the sample trajectories; and
storing the agent as machine trained.

14. The method of claim 13, wherein the medical sensor comprises an ultrasound transducer, and wherein machine training comprises training the agent to output the movement to position the ultrasound transducer aₜ an imaging goal.

15. The method of claim 13, wherein obtaining comprises obtaining from simulation.

16. The method of claim 15, wherein machine training comprises machine training the agent using pairs of input trajectories sampled from different patients for a given iteration in an optimization.

17. The method of claim 13, wherein machine training comprises machine training with the contrastive reinforcement learning framework comprising a policy network implementing the agent trained on a critic loss where the critic loss is from a critic network operating on a first latent representation for a goal and a second latent representation for a state-action pair, the state-action pair based on the movement.

18. The method of claim 17, wherein machine training comprises machine training with the critic network is trained to maximize similarity between the state-action pair and the goal and minimize similarity when sampled from a different trajectory.

19. A system for medical sensor navigation, the system comprising:
a memory configured to store a policy network machine trained in a contrastive reinforcement learning framework;
an input configured to receive a goal for medical imaging; and
a processor configured to output an action to move the medical sensor towards the goal based on input of the goal and a current state of the medical sensor to the policy network, which policy network outputs the action in response to the input.

20. The system of claim 19 wherein the policy network was trained using trajectories with inputs sampled from different patients for a same iteration in optimization of a critic network of the contrastive reinforcement learning framework, the trajectories created from simulation.
